(19) United States
(12) Patent Application Publication
(10) Pub. No.: US 2008/0086201 A1
(43) Pub. Date: Apr. 10, 2008

(54) MAGNETIZED BIOERODIBLE ENDOPROSTHESIS

(75) Inventors: Jan Weber, GJ, Maastrich (NL); Liliana Atanasoska, Edina, MN (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/855,472

(22) Filed: Sep. 14, 2007

Related U.S. Application Data
(60) Provisional application No. 60/844,832, filed on Sep. 15, 2006.

(51) Int. Cl. A61F 2/06

(52) U.S. Cl. 623/1.42; 424/426

(57) ABSTRACT

Endoprostheses (e.g., stents) having a magnetized portion and a bioerodible portion are disclosed.

### CROSS-REFERENCE TO RELATED APPLICATIONS

This application claims priority under 35 USC §119(e) to U.S. Provisional Patent Application Ser. No. 60/844,832, filed on Sep. 15, 2006, the entire contents of which are hereby incorporated by reference.

### TECHNICAL FIELD

This invention relates to medical devices, such as endoprostheses, and methods of making and using the same.

### BACKGROUND

The body includes various passageways including blood vessels such as arteries, and other body lumens. These passageways sometimes become occluded or weakened. For example, they can be occluded by a tumor, restricted by plaque, or weakened by an aneurysm. When this occurs, the passageway can be reopened or reinforced, or even replaced, with a medical endoprosthesis. An endoprosthesis is an artificial implant that is typically placed in a passageway or lumen in the body. Many endoprostheses are tubular members, examples of which include stents, stent-grafts, and covered stents.

Many endoprostheses can be delivered inside the body by a catheter. Typically the catheter supports a reduced-size or compacted form of the endoprosthesis as it is transported to a desired site in the body, for example the site of weakening or occlusion in a body lumen. Upon reaching the desired site the endoprosthesis is installed so that it can contact the walls of the lumen.

One method of installation involves expanding the endoprosthesis. The expansion mechanism used to install the endoprosthesis may include forcing it to expand radially. For example, the expansion can be achieved with a catheter that carries a balloon in conjunction with a balloon-expandable endoprosthesis reduced in size relative to its final form in the body. The balloon is inflated to deform and/or expand the endoprosthesis in order to fix it at a predetermined position in contact with the lumen wall. The balloon can then be deflated, and the catheter withdrawn.

In another delivery technique, the endoprosthesis is formed of an elastic material that can be reversibly compacted and expanded (e.g., elastically or through a reversible phase transition of its constituent material). Before and during introduction into the body until it reaches the desired implantation site, the endoprosthesis is restrained in a compacted condition. Upon reaching the desired site, the restraint is removed, for example by retracting a restraining device such as an outer sheath, enabling the endoprosthesis to self-expand by its own internal elastic restoring force.

To support or keep a passageway open, endoprostheses are sometimes made of relatively strong materials, such as stainless steel or Nitinol (a nickel-titanium alloy), formed into struts or wires. The material from which an endoprosthesis is made can impact not only the way in which it is installed, but its lifetime and efficacy within the body.

### SUMMARY

In one aspect, the invention features an endoprosthesis, e.g., a stent, that includes a magnetized portion and a bioerodible portion.

In another aspect, the invention features a method of implanting an endoprosthesis (e.g., a stent) having a magnetized portion and a bioerodible portion (e.g., an endoprostheis as described herein) in a body passageway of an organism. The endoprosthesis can be magnetized prior to, during, or after, delivery into the body. The magnetization of the endoprosthesis can be varied after delivery into the body.

In yet another aspect, the invention features a method of delivering an endoprosthesis, e.g., stent, into the vascular system. The method includes delivering the endoprosthesis, e.g., stent, through a lumen utilizing an elongated delivery device; the delivery device can include one or more elements magnetically attracted to the endoprosthesis, e.g., stent. In some embodiments, the magnetic element is moveable relative to the endoprosthesis, e.g., stent. In other embodiments, the delivery device used includes a balloon catheter. In yet other embodiments, the catheter includes the magnetic element. The delivery device can further include a guidewire.

In a further aspect, the invention features a method of making an endoprosthesis, e.g., stent. The method includes forming an endoprosthesis having a magnetized or magnetizeable portion and/or a bioerodible portion, and optionally, magnetizing the magnetizeable portion, e.g., by applying a magnetic field or a current.

Embodiments may include one or more of the following features. The magnetized portion can be bioerodible. The entire endoprosthesis, e.g., stent, is bioerodible and/or magnetized. The endoprosthesis, e.g., stent, has a magnetic field of about 0.001 Tesla or more, typically 0.005 Tesla or more. The endoprothesis, e.g., stent, has a bioerodible portion that includes a metal. The endoprothesis, e.g., stent, has a magnetized portion that includes a ferromagnetic metal, a paramagnetic metal, a lanthanoid, or a mixture thereof. The ferromagnetic metal can be chosen from, e.g., one or more of iron, nickel, manganese or cobalt. The paramagnetic metal can be chosen from, e.g., one or more of magnesium, molybdenium, lithium or tantalum. The bioerodible portion is a polymer, e.g., a polymer chosen from one or more of: polyiminocarbonates, polycarbonates, polyarylates, polylactides, or polyglycolic esters. The polymer includes a magnetizeable material. The magnetizeable material can be provided, for example, as a coating on the polymer, or within a polymer body. The endoprosthesis, e.g., stent, includes a non-bioerodible portion. The non-bioerodible portion can be magnetized. The non-bioerodible portion includes a bioerodible coating (e.g., a coating that includes a polymer, an inorganic material (e.g., an oxide or silica) or a metal).

Embodiments may further include one or more of the following features. The endoprosthesis, e.g., stent, can further include at least one therapeutic agent or drug. The therapeutic agent can be chosen from, e.g., one or more of: an anti-thrombogenic agent, an anti-proliferative/anti-mitotic agents, an inhibitor of smooth muscle cell proliferation, an antioxidant, an anti-inflammatory agent, an anesthetic agents, an anti-coagulant, an antibiotic, and an agent that stimulates endothelial cell growth and/or attachment. The therapeutic agent is paclitaxel. The therapeutic agent can be present in one or more magnetic capsules.

Embodiments may also include one or more of the following features. Magnetization is controlled to modulate the erosion rate and/or endothelialization. In other embodiments, the endoprosthesis, e.g., stent, carries a therapeutic agent (e.g., a drug) and embodiments include controlling magnetization to control drug delivery.

Aspects and/or embodiments may have one or more of the following additional advantages. The endoprostheses may not need to be removed from a lumen after implantation. The endoprostheses can have low thrombogenecity. Lumens implanted with the endoprostheses, particularly, the magnetized portion of the endoprosthesis, can exhibit reduced restenosis. The magnetized portions of the endoprosthesis can support cellular growth (endothelialization). The rate of release of a therapeutic agent from an endoprosthesis can be controlled. The rate of bioerosion of different portions of the endoprostheses can be controlled, thus allowing the endoprostheses to erode in a predetermined manner, as well as reducing and/or localizing the fragmentation. For example, magnetized portions of the endoprosthesis, e.g., stent, can erode at a faster rate that the non-magnetized regions. Eroded fragments can remain localized to the endoprosthesis due to magnetic forces. Stent securement can be facilitated (e.g., by embedding magnetic elements in the stent delivery device). Furthermore, drug delivery from the endoprosthesis can be improved (e.g., by attaching magnetic drug delivery capsules to the endoprosthesis, and/or controlling drug release).

An erodible or bioerodible medical device, e.g., a stent, refers to a device, or a portion thereof, that exhibits substantial mass or density reduction or chemical transformation, after it is introduced into a patient, e.g., a human patient. Mass reduction can occur by, e.g., dissolution of the material that forms the device and/or fragmenting of the device. Chemical transformation can include oxidation/reduction, hydrolysis, substitution, electrochemical reactions, addition reactions, or other chemical reactions of the material from which the device, or a portion thereof, is made. The erosion can be the result of a chemical and/or biological interaction of the device with the body environment, e.g., the body itself or body fluids, into which it is implanted and/or erosion can be triggered by applying a triggering influence, such as a chemical reactant or energy to the device, e.g., to increase a reaction rate. dFor example, a device, or a portion thereof, can be formed from an active metal, e.g., Mg or Ca or an alloy thereof, and which can erode by reaction with water, producing the corresponding metal oxide and hydrogen gas (a redox reaction). For example, a device, or a portion thereof, can be formed from an erodible or bioerodible polymer, or an alloy or blend erodible or bioerodible polymers which can erode by hydrolysis with water. The erosion occurs to a desirable extent in a time frame that can provide a therapeutic benefit. For example, in embodiments, the device exhibits substantial mass reduction after a period of time which a function of the device, such as support of the lumen wall or drug delivery is no longer needed or desirable. In particular embodiments, the device exhibits a mass reduction of about 10 percent or more, e.g. about 50 percent or more, after a period of implantation of one day or more, e.g. about 60 days or more, about 180 days or more, about 600 days or more, or 1000 days or less. In embodiments, the device exhibits fragmentation by erosion processes. The fragmentation occurs as, e.g., some regions of the device erode more rapidly than other regions. The faster eroding regions become weakened by more quickly eroding through the body of the endoprosthesis and fragment from the slower eroding regions. The faster eroding and slower eroding regions may be random or predefined. For example, faster eroding regions may be predefined by treating the regions to enhance chemical reactivity of the regions. Alternatively, regions may be treated to reduce erosion rates, e.g., by using coatings. In embodiments, only portions of the device exhibits erodibilty. For example, an exterior layer or coating may be erodible, while an interior layer or body is non-erodible. In embodiments, the endoprosthesis is formed from an erodible material dispersed within a non-erodible material such that after erosion, the device has increased porosity by erosion of the erodible material.

Erosion rates can be measured with a test device suspended in a stream of Ringer's solution flowing at a rate of 0.2 m/second. During testing, all surfaces of the test device can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter.

All publications, patent applications, patents, and other references mentioned herein are incorporated by reference herein in their entirety.

Other aspects, features, and advantages will be apparent from the description and drawings, and from the claims.

### DESCRIPTION OF DRAWINGS

FIGS. 1A-1C are views of a bioerodible stent. FIG. 1A is a perspective view of the stent. FIGS. 1B and 1C are expanded schematic views of the circled section of the stent of FIG. 1A.

FIGS. 2A-2E are longitudinal cross-sectional views, illustrating delivery of a magnetized bioerodible stent in a collapsed state (FIG. 2A), expansion of the stent (FIGS. 2B-C) and deployment of the stent (FIG. 2D). FIG. 2E depicts the process of erosion showing the enhanced localization of the stent fragments by the magnetic field.

FIG. 3 is a cross section through an embodiment of a stent.

FIGS. 4A-4C are cross-sectional views of magnetized capsules containing one or more therapeutic agents.

FIG. 5 is a perspective view of a method of magnetizing a bioerodible stent using a solenoid.

Like reference symbols in the various drawings indicate like elements.

### DETAILED DESCRIPTION

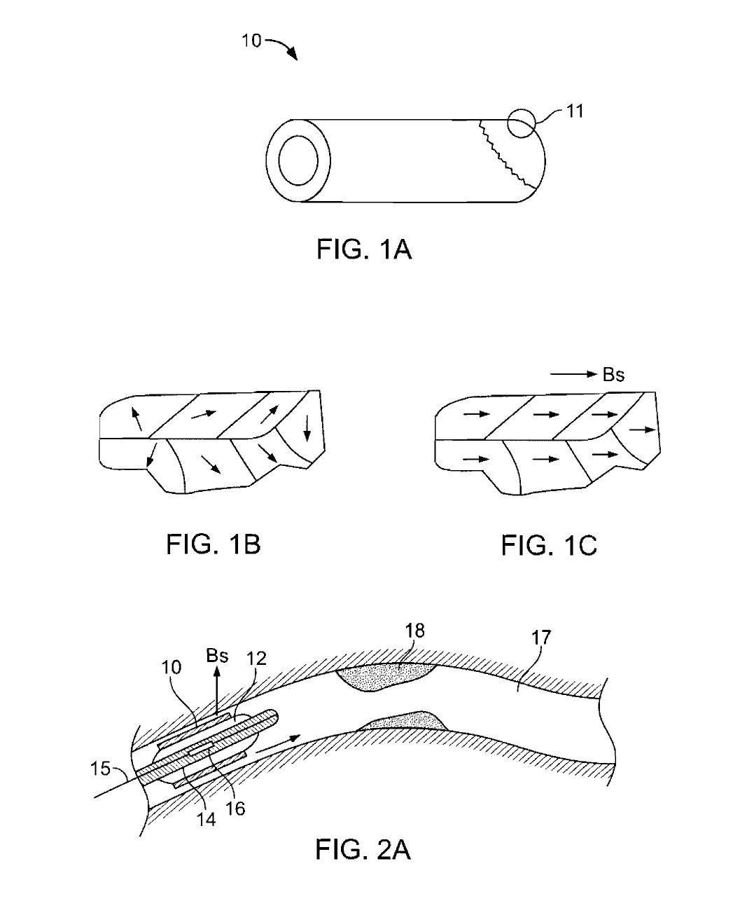

Referring to FIG. 1A, an exemplary device 10 is generally tubular in shape and as depicted may be, e.g., a stent. Referring as well to FIGS. 1B and 1C, depicted are two expanded schematic views of a magnetizeable portion 11 of the exemplary device 10, illustrating the electron spin of the magnetizeable domains before and after becoming magnetized, respectively. In embodiments shown in FIGS. 1A-1C, the magnetizable portion is part of the body of the stent (e.g., the stent is formed in selected portions or entirely out of the magnetizeable material). The magnetizeable portion 11 is depicted in a non-magnetized state in FIG. 1B by showing the electron spins (arrows) in a relative random orientation and the net magnetic field for the part as a whole is about zero. The magnetizeable portion 11 becomes magnetized by applying a magnetizing force, e.g., by applying an external magnetic field to, or by passing an electrical current through, the material. Application of the magnetizing force leads to the alignment of the electron spins in the magnetizable portion 11 in a substantially unidirectional configuration as depicted by the arrows pointing to one orientation in FIG. 1C, thereby producing a magnetic pole (Bs). The magnetizeable portion 11 is in a magnetized state when the atoms within the material carry a magnetic moment and the material includes regions known as magnetic domains. In each magnetic domain, the atomic dipoles are coupled together in substantially the same direction. Some or all of the domains can become aligned. The more domains that are aligned, the stronger the magnetic field in the material. When all of the domains are aligned, the material is considered to be magnetically saturated. Magnetization of the erodible stent can enhance erosion in the body, reduce the likelihood that large fragments resulting from erosion will enter the bloodstream, reduce restenosis by enhancing endothelial growth on outer surfaces of the stent while reducing smooth muscle growth, and enhanced deliverability.

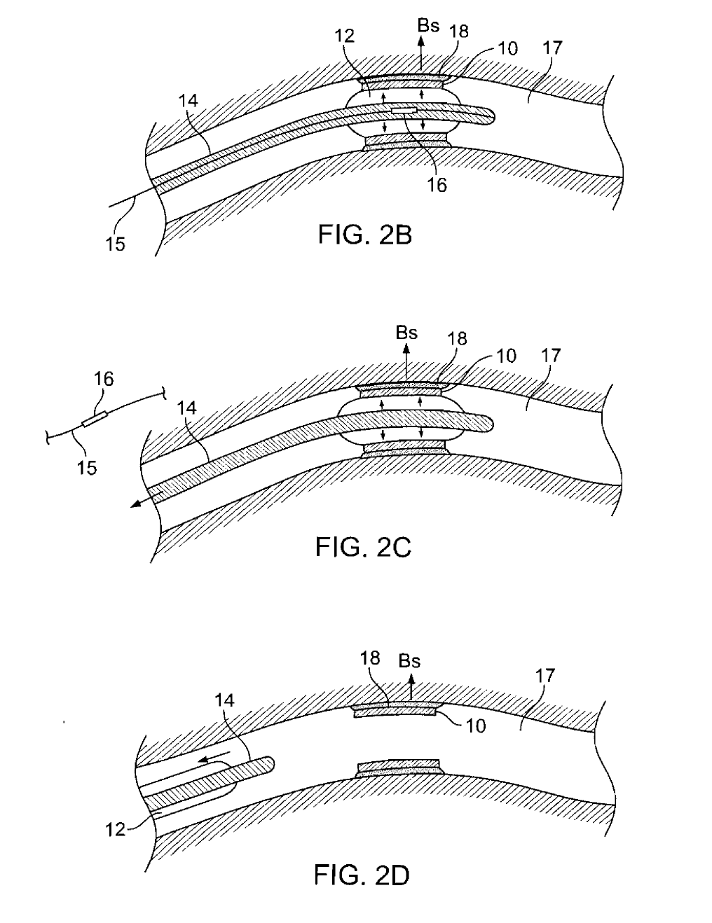

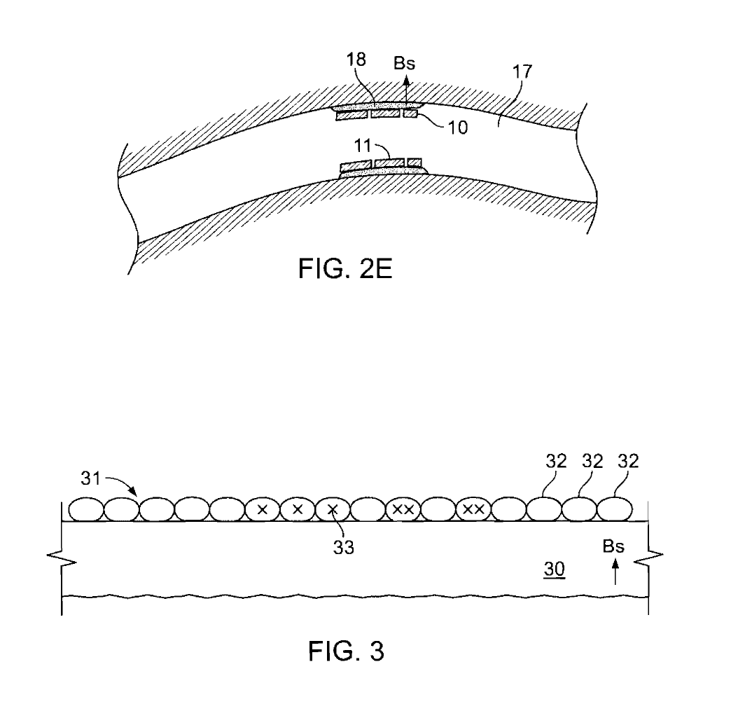

Referring to FIGS. 2A-2E, a magnetized bioerodible stent 10 with a magnetic pole (Bs) is placed over a balloon 12 carried near the distal end of a catheter 14, and is directed through a lumen 17 (FIG. 2A) until the portion carrying the balloon and stent reaches the region of an occlusion 18. The stent 10 is then radially expanded by inflating the balloon 12 and pressed against the vessel wall with the result that occlusion 18 is compressed, and the vessel wall surrounding it undergoes a radial expansion (FIG. 2B). A catheter or wire 15, e.g., a guidewire, containing one more magnetic elements 16 can, optionally, be inserted inside the catheter 14 and positioned such that the magnetic elements 16 are located within the balloon and the stent (FIG. 2C). The magnetic attraction forces between the stent and the elements enhance the securement of the stent on the balloon, reducing dislodgement of the stent or chafing between the balloon and the stent as the system is delivered into the body lumen. When the location of stent deployment is reached, the catheter or wire containing the one or more magnetized elements can be removed from the catheter 14 to facilitate release of the stent when the balloon is inflated (FIG. 2C). The pressure is then released from the balloon and the catheter 14 is withdrawn from the vessel (FIG. 2D). In other embodiments, magnetic elements may be mounted on the catheter 14 and the attractive force between the magnetic elements and the stent can be overcome by expansion of the balloon. In other embodiments, the magnetic elements are present on the balloon 12. In embodiments, the magnetization of the elements can be reduced or eliminated before, during or after stent deployment. Referring to FIG. 2E, over time, the stent 10 erodes in the body, sometimes creating fragments 11. The field Bₛ attracts the fragments to each other, reducing the risk that the fragments will be dislodged from the body lumen wall and enter the bloodstream. In addition, the field Bₛ encourages endothelial growth from the lumen wall which envelopes the stent and also discourages dislodgement of the fragments.

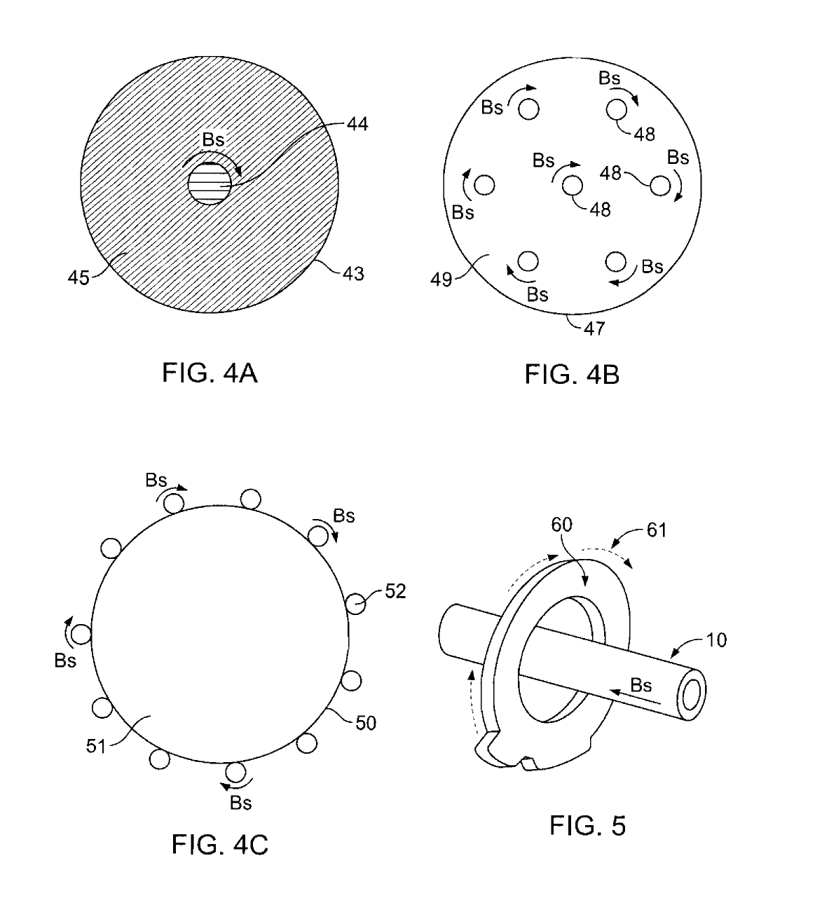

Referring to FIG. 3, a cross section through a stent wall 30, in embodiments, the stent includes a coating 31 that carries and releases a drug 33. The coating 31 can be formed by a series of capsules 32 that are magnetically attracted to the stent body. Referring to FIGS. 4A-4C, cross-sectional views of three embodiments of magnetized capsules containing one or more therapeutic agents are illustrated. Referring particularly to FIG. 4A, in embodiments, a capsule 43 includes a magnetic particle 44 coated with a polymer 45 incorporating a therapeutic agent. Alternatively, the therapeutic agent can be coated directly on the magnetic particle. The particle 44 is magnetically attached to the stent body, thus securing the capsule to the stent body during use. Suitable particles include ferromagnetic materials, e.g. iron. Suitable polymers include nonbioerodible, drug eluting polymers, e.g., styrene-isobutylene-styrene (SIBs); and bioerodible polymers, e.g., having a biocompatible coating such as a lipid or phospholipid. Suitable drug-containing polymers are described in U.S. patent Appln. No. 2005/0192657. Referring to FIG. 4B, in embodiments, a capsule 47 is provided with magnetic material 48 dispersed through a polymer 49. Referring as well to FIG. 4C, in embodiments, a capsule 50 includes a polymer 51 incorporating a drug, and a magnetic material 52 provided as a layer on the particles. The layer is interrupted at locations to allow drug to elute from the polymer. In embodiments, the capsules are sized to facilitate absorption by the body over time. For example, in embodiments, the capsules have a diameter of about 50 nm to 100 micrometer, e.g., about 100 nm to 30 micrometer. In other embodiments, the magnetic material may be provided in a uniform polymer layer applied to the stent body, which optionally carries a drug. In embodiments, the magnetizeable, biocrodible stent includes a coating of a drug or a polymer, including a drug without magnetic material.

Referring to FIG. 5, the stent 10, and/or the particles, can be magnetized before or after delivery into the body. Magnetization can be performed by applying an external magnetic field provided by a solenoid 60. The stent 10 is placed in any direction, e.g., longitudinally or perpendicularly, in a concentrated magnetic field that fills the center of the solenoid 60. A current, e.g., a DC current, 61 is passed through the solenoid to generate the magnetic field. Other sources of magnetic field that can be used include a coil or a magnet (e.g., a permanent magnet or, typically, an electromagnet). In other embodiments, the stent is magnetized by direct exposure to a current. In those embodiments where the endoprosthesis is magnetized inside an organism, e.g., a patient, a non-magnetized stent is implanted in a selected passageway of the organism; the organism is then exposed to a magnetic field generated by, e.g., a solenoid chamber. The magnetic field can be localized to the area where the endoprosthesis has been implanted, e.g., the chest. In one embodiment, a small diameter solenoid having a plurality of coils is used. A high current is applied on both sides of the body such that they are positioned along the same axis with the endoprosthesis somewhere in the middle point. The strength of magnetization can also be reduced by, e.g., exposing the endoprosthesis to an AC field. The degree of magnetization can be controlled to facilitate delivery, drug elution and erosion.

In certain embodiments, permanent magneticity (retentivity) can be induced inside a body. In such embodiments, a strong magnet, e.g., a Neodynium magnet, can be brought in close proximity to the ferromagnetic material, e.g., iron. Iron is typically used as it readily magnetizes. For example, if a piece of iron is brought near a permanent magnet, the electrons within the atoms in the iron orient their spins to match the magnetic field force produced by the permanent magnet, and the iron becomes “magnetized.” Iron will typically magnetize in such a way as to incorporate the magnetic flux lines into its shape, which attracts it toward the permanent magnet, regardless of which pole of the permanent magnet is offered to the iron. The previously unmagnetized iron becomes magnetized as it is brought closer to the permanent magnet. No matter what pole of the permanent magnet is extended toward the iron, the iron will typically magnetize in such a way as to be attracted toward the magnet. The strong magnet can be positioned on a catheter that is delivered to the site at which the endoprosthesis is implanted. The strong magnet can also be located outside the body at a position corresponding to the implanted stent. A strong magnet can also be used to magnetize an endoprosthesis prior to delivery into the body.

The degree of magnetization typically decreases as the ferromagnetic material (e.g., iron) corrodes. In some embodiments, the endoprosthesis, e.g., stent, can be coated with a corrosion protection layer, e.g., a layer that includes iron nitride, which still allows the endoprosthesis, e.g., stent, to be magnetized, but can act as a protection layer to reduce the rate of corrosion (Chattopadhyay, S. K. et al. (1998) Solid State Communications, Vol. 108, No. 12: 977-982).

Magnetization of ferromagnetic materials can be measured in several ways known in the art. For example, a Hall sensor (e.g., a one-dimensional, two- and even three-dimensional Hall sensor) can be used. Hall sensors are commercially available, e.g., from Sentron in Switzerland. Another way of measuring magnetization is to use magnetic force microscopy. Generally, in a magnetic force microscope, a magnetic tip is used to probe the magnetic stray field above a sample surface. The magnetic tip is typically mounted on a small cantilever that translates the force into a deflection which can be measured. The microscope can sense the deflection of the cantilever which results in an image, e.g., a force image (static mode) or a resonance frequency change of the cantilever that results in a force gradient image. The sample can be scanned under the tip, which results in mapping of the magnetic forces or force gradients above the surface. Magnetic force microscopy allows to map the entire surface of the endoprosthesis, e.g., stent, to determine whether certain areas of the endoprosthesis are more or less magnetic. See, Sandhu, A. et al. (2001) Jpn. J. Appl. Phys. Vol. 40:4321-4324; Part 1, No. 6B, for an example of magnetic imaging by scanning Hall probe microscopy.

In embodiments, the stent is formed of a material or combination of materials such that at least portions of the stent are bioerodible and portions are magnetizeable. Suitable magnetizeable materials include ferromagnetic and paramagnetic materials. In those embodiments where a paramagnetic material is used, a permanent magnet or magnetic field is typically placed in the vicinity of the material to keep the substrate magnetized. For example, an endoprosthesis, e.g., stent, can have a portion that includes a permanent magnet and a portion that includes a paramagnetic material. Suitable magnetizeable metals include iron, nickel, manganese and cobalt. In those embodiments where cobalt is used, it is typically embedded within a non-bioerodible material (e.g., within a non-bioerodible portion of the stent or coating) to minimize exposure of cobalt to the body. In other embodiments, the endoprosthesis, e.g., stent, has a portion that includes one or more rare earth elements (e.g., lanthanoids). For example, one or more rare earth elements can form an alloy and be magnetized to produce a strong magnetic field.

The bioerodible material can be a bioerodible metal, a bioerodible metal alloy, or a bioerodible non-metal. Bioerodible materials are described, for example, in U.S. Pat. No. 6,287,332 to Bolz; U.S. patent Application Publication No. US 2002/0004060 A1 to Heublein; U.S. Pat. Nos. 5,587,507 and 6,475,477 to Kohn et al. Examples of bioerodible metals include alkali metals, alkaline earth metals (e.g., magnesium), iron, zinc, and aluminum. Examples of bioerodible metal alloys include alkali metal alloys, alkaline earth metal alloys (e.g., magnesium alloys), iron alloys (e.g., alloys including iron and up to seven percent carbon), zinc alloys, and aluminum alloys. Examples of bioerodible non-metals include bioerodible polymers, such as, e.g., polyanhydrides, polyorthoesters, polylactides, polyglycolides, polysiloxanes, cellulose derivatives and blends or copolymers of any of these. Bioerodible polymers are disclosed in U.S. Published patent Application No. 2005/0010275, filed Oct. 10, 2003; U.S. Published patent Application No. 2005/0216074, filed Oct. 5, 2004; and U.S. Pat. No. 6,720,402.

The magnetizeable portion and the bioerodible portion can be combined in various arrangements. In embodiments, the body of the stent is formed entirely out of a material that is both bioerodible and magnetizeable. A suitable material is iron. In other embodiments, the stent body is formed of a nonmagnetizeable bioerodible material that includes within its matrix or as a coating a magnetizeable material. The nonmagnetizeable bioerodible material may be, for example, an inorganic material, a metal, a polymer, or a ceramic. For example, the stent body may be made of a bioerodible polymer. The polymer may include magnetizeable particles embedded within the polymer matrix and/or a layer of magnetizeable material may be coated on or provided within the polymer body to form a composite structure. In some embodiments, only portions of the endoprosthesis are erodible. For example, an exterior layer or coating may be eroded, while an interior layer or body is non-erodible. In embodiments, the endoprosthesis is formed from an erodible material dispersed within a non-erodible material such that after erosion, the endoprosthesis has increased porosity. The increased porosity results at least in part from the erosion of the erodible material.

In other embodiments, the stent can include one or more biostable and/or non-magnetizeable or magnetizeable materials in addition to one or more bioerodible and magnetizeable materials. For example, the bioerodible material and the magnetizeable material may be provided as a coating on a biostable and non-magnetizeable stent body. Examples of biostable materials include stainless steel, tantalum, nickel-chrome, cobalt-chromium alloys such as Elgiloy® and Phynox®, Nitinol (e.g., 55% nickel, 45% titanium), and other alloys based on titanium, including nickel titanium alloys, thermo-memory alloy materials. Stents including biostable and bioerodible regions are described, for example, in commonly owned U.S. Patent Application Publication No. 2006-0122694 A1, entitled “Medical Devices and Methods of Making the Same.” The material can be suitable for use in, for example, a balloon-expandable stent, a self-expandable stent, or a combination of both (see e.g., U.S. Pat. No. 5,366,504). The components of the medical device can be manufactured, or can be obtained commercially. Methods of making medical devices such as stents are described in, for example, U.S. Pat. No. 5,780,807, and U.S. patent Application Publication No. 2004-0000046-A1, both of which are incorporated herein by reference. Stents are also available, for example, from Boston Scientific Corporation, Natick, Mass., USA, and Maple Grove, Minn., USA.

Restenosis reduction or prevention and the erosion rate can be controlled by controlling the strength of magnetization. The effect of magnetization on restenosis is discussed in Lu et al, Chin Med J 2001; 114(8): 831-823. Magnetized materials have been shown to corrode in solution at a faster rate than non-magnetized samples (Costa, I. et al. (2004) Journal of Magnetism and Magnetic Materials 278:348-358). Without being bound by theory, the faster erosion rate of the magnetized portion is believed to relate to the effect of the magnetic field on the oxygen transport from solution to the magnet surface. Since oxygen molecules are paramagnetic, their transport towards the electrode surface is believed to be affected by the magnetic field. It is proposed that the oxygen transport to the interface of the magnet and electrolyte is facilitated by the magnetic field, which leads to an increase supply of oxidizing species to the interface and consequently accelerating the charge transfer phenomena that ultimately leads to the erosion of the magnetized portion. In some embodiments, the magnetized portion erodes, e.g., inside an organism, at a faster rate than the corresponding non-magnetized material. For example, the magnetized portion can erode at a rate 1.5, 2, 3, 4, 5, 6-fold, or higher than the corresponding non-magnetized material. Erosion rates can be measured with a test endoprosthesis suspended in a stream of Ringer's solution flowing at a rate of 0.2 m/second. During testing, all surfaces of the test endoprosthesis can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter. Experimental conditions for testing erosion/erosion rates of magnetized versus non-magnetized samples are disclosed in Costa, I. et al. (2004) supra. For example, the rates of erosion can be measured using naturally aerated 3.5% by weight NaCl solution. Electrochemical and weight loss measurements can be measured as described by Costa, I. et al. (2004) supra. In embodiments, the stent exhibits a magnetic field strength of about 0.001 Tesla or more, e.g., 0.005 Tesla or more.

A therapeutic agent can be carried by the endoprosthesis (e.g., stent), e.g., dispersed within a bioerodible and/or magnetized portion of the stent, or dispersed within an outer layer of the stent (e.g., a coating). The therapeutic agent can also be carried exposed surfaces of the stent. The terms “therapeutic agent,” “pharmaceutically active agent,” “pharmaceutically active material,” “pharmaceutically active ingredient,” “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074, the entire disclosure of which is hereby incorporated by reference herein.

Medical devices, in particular endoprostheses, including at least a portion being magnetized, bioerodible as described above include implantable or insertable medical devices, including catheters (for example, urinary catheters or vascular catheters such as balloon catheters), guide wires, balloons, filters (e.g., vena cava filters), stents of any desired shape and size (including coronary vascular stents, aortic stents, cerebral stents, urology stents such as urethral stents and ureteral stents, biliary stents, tracheal stents, gastrointestinal stents, peripheral vascular stents, neurology stents and esophageal stents), grafts such as stent grafts and vascular grafts, cerebral aneurysm filler coils (including GDC-Guglilmi detachable coils- and metal coils), filters, myocardial plugs, patches, pacemakers and pacemaker leads, heart valves, and biopsy devices. Indeed, embodiments herein can be suitably used with any underlying structure (which can be, for example, metallic, polymeric or ceramic, though preferably metallic) which is coated with a fiber meshwork in accordance with methods herein and which is designed for use in a patient, either for procedural use or as an implant.

The medical devices may further include drug delivery medical devices for systemic treatment, or for treatment of any mammalian tissue or organ. Subjects can be mammalian subjects, such as human subjects (e.g., an adult or a child). Non-limiting examples of tissues and organs for treatment include the heart, coronary or peripheral vascular system, lungs, trachea, esophagus, brain, liver, kidney, bladder, urethra and ureters, eye, intestines, stomach, colon, pancreas, ovary, prostate, gastrointestinal tract, biliary tract, urinary tract, skeletal muscle, smooth muscle, breast, cartilage, and bone.

In some embodiments, the medical device, e.g., endoprosthesis, is used to temporarily treat a subject without permanently remaining in the body of the subject. For example, in some embodiments, the medical device can be used for a certain period of time (e.g., to support a lumen of a subject), and then can disintegrate after that period of time.

The medical device, e.g., endoprosthesis, can be generally tubular in shape and can be a part of a stent. Simple tubular structures having a single tube, or with complex structures, such as branched tubular structures, can be used. Depending on specific application, stents can have a diameter of between, for example, 1 mm and 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stents can also be preferably bioerodible, such as a bioerodible abdominal aortic aneurysm (AAA) stent, or a bioerodible vessel graft.

All publications, patent applications, patents, and other references mentioned herein are incorporated by reference herein in their entirety.

Other embodiments are within the scope of the following claims.

What is claimed is:

1. A stent comprising a magnetized portion and a bioerodible portion.

2. The stent of claim 1, wherein the magnetized portion is bioerodible.

3. The stent of claim 1, wherein the entire stent is bioerodible.

4. The stent of claim 1, wherein the entire stent is magnetized.

5. The stent of claim 1, wherein the bioerodible portion is a metal.

6. The stent of claim 1, wherein the magnetized portion comprises a ferromagnetic metal, a paramagnetic metal, or a mixture thereof.

7. The stent of claim 6, wherein the ferromagnetic metal is selected from the group consisting of iron, nickel, manganese and cobalt.

8. The stent of claim 6, wherein the paramagnetic metal is selected from the group consisting of magnesium, molybdenium, lithium and tantalum.

9. The stent of claim 1, wherein the bioerodible portion is a polymer.

10. The stent of claim 9, wherein the polymer is selected from the group consisting of polyiminocarbonates, polycarbonates, polyarylates, polylactides, and polyglycolic esters.

11. The stent of claim 1, wherein the polymer includes a magnetizeable material.

12. The stent of claim 11, wherein the magnetizeable material is provided as a coating on the polymer.

13. The stent of claim 11, wherein the magnetizeable material is provided within a polymer body.

14. The stent of claim 1, including a nonbioerodible portion.

15. The stent of claim 14, wherein the nonbioerodible portion is magnetized.

16. The stent of claim 14 or 15, wherein the nonbioerodible portion includes a bioerodible coating.

17. The stent of claim 16, wherein the coating is a polymer.

18. The stent of claim 16, wherein the coating is an inorganic material.

19. The stent of claim 16, wherein the coating is a metal.

20. The stent of claim 1, further comprising at least one therapeutic agent.

21. The stent of claim 20, wherein the at least one therapeutic agent is selected from the group consisting of chosen an anti-thrombogenic agent, an anti-proliferative/anti-mitotic agents, an inhibitor of smooth muscle cell proliferation, an antioxidant, an anti-inflammatory agent, an anesthetic agents, an anti-coagulant, an antibiotic, and an agent that stimulates endothelial cell growth and/or attachment.

22. The stent of claim 20, wherein the at least one therapeutic agent is paclitaxel.

23. The stent of claim 1, wherein at least one therapeutic agent is present in one or more magnetic capsules.

24. The stent of claim 1, wherein the stent has a magnetic field of about 0.001 Tesla or more.

25. A method comprising implanting the stent of claim 1 in a body passageway of an organism.

26. The method of claim 25 comprising magnetizing the stent prior to delivery into the body.

27. The method of claim 25 comprising magnetizing the stent after delivery into the body.

28. The method of any one of claims 25 to 27 comprising varying the magnetization of the stent after delivery into the body.

29. The method of claim 28 comprising controlling magnetization to control erosion rate.

30. The method of claim 28 comprising controlling magnetization to control endothelialization.

31. The method of claim 28 wherein the stent carries a therapeutic agent and controlling magnetization to control drug delivery.

32. The method of claim 25 wherein the stent carries a drug.

33. The method of claim 25 comprising delivering the stent into the vascular system.

34. The method of claim 25 comprising delivering said stent through a lumen utilizing an elongated delivery device, the delivery device including an element magnetically attracted to the stent.

35. The method of claim 34 wherein the magnetic element is moveable relative to the stent.

36. The method of claims 33 or 34 wherein the delivery device includes a balloon catheter.

37. The method of claim 35 wherein the catheter includes said magnetic element.

38. The method of claims 33 or 34 wherein the delivery device includes a guidewire.
